(19) United States
(12) Patent Application Publication
(10) Pub. No.: US 2013/0079624 A1
(43) Pub. Date: Mar. 28, 2013

(54) GRAPHICAL USER INTERFACE FOR OPERATING AN MRI

(71) Applicant: Uri RAPOPORT, Moshav Ben Shemen (IL)

(72) Inventor: Uri RAPOPORT, Moshav Ben Shemen (IL)

(21) Appl. No.: 13/241,393

(22) Filed: Sep. 23, 2011

(51) Int. Cl. A61B 5/055

(52) U.S. Cl. 600/410

(57) ABSTRACT

A method for scanning using an MRI, including: providing a display, MRI, object to be scanned in said MRI, and a GUI; connecting said display with said MRI; displaying said GUI on said display; displaying at at least one first region of said GUI, graphic visualization options relating to said object and scanning of object, said visualizations are selected from a group consisting of type of object, object orientation, preferred scanning region of object, preferred scanning quality of object, preferred scanning orientation, preferred scanning type, and a combination thereof; selecting said object characteristics and scanning characteristics via said GUI; scanning said object by means of said MRI in accordance with said determined characteristics; and optionally, selecting a presentation mode of said scanning from a group consisting of single image display, multi-image display, thumbnail display, comparative display and a combination thereof, and displaying the same on said display.

### FIELD OF THE INVENTION

The present invention generally pertains to the field of MRI operation. In particular, it relates to means and method for operating a user-friendly GUI adapted for controlling MRI parameters and viewing MRI scans.

### BACKGROUND

Operation of MRI is performed today by directly controlling the MRI parameters; therefore, an MRI user must be a person with extensive knowledge of the physics of MRI. Often, MRI users are researchers which have not been trained to operate an MRI and are in need to be able to control the MRI scanning by a simple non-technical language. In addition, handling the images produced by the MRI is done in interfaces which are not intuitive and are limited in their options.

U.S. Pat. No. 5,606,258 describes a controlling system for operating MRI, however, this controlling system still requires the user knowledge of the MRI structure. US patent application 20100037182 describes a user interface for displaying MRI images. However, the user interface is adapted to provide view of single scans and is nor adapted for more complicated requirement such as scans compare.

There is therefore a long unmet need for a user friendly interface which does not require any technical background for operating an MRI and handling MRI scans.

### BRIEF SUMMARY

An object of the invention is thus to disclose a method for scanning using an MRI. Method is characterized by steps of (i) providing a display, an MRI, an object to be scanned in said MRI, and a GUI; (ii) connecting a display with an MRI; (iii) displaying a GUI on a display; (iv) displaying at at least one first region of a GUI, graphic visualization options relating to an object and to scanning of object, visualizations are selected from a group consisting of type of object, object orientation, preferred scanning region of object, preferred scanning quality of object, preferred scanning orientation, preferred scanning type, and a combination thereof; (v) selecting object characteristics and scanning characteristics via said GUI; (vi) scanning an object by means of an MRI in accordance with the determined characteristics; (vii) selecting a presentation mode of the scanning from a group consisting of single image display, multi-image display, thumbnail display, comparative display and a combination thereof; (viii) displaying the same on said display.

It is another object of the invention to disclose the method as defined above, additionally comprising step of selecting the type of object that we are working with according to the standard biological classification of organisms from a group consisting of kingdom, phylum, class, order, family, genus, species, subspecies and a combination thereof.

It is another object of the invention to disclose the method as defined above, additionally comprising step of selecting a type of object from a group consisting of object identification number, type of experiment, type of disease, and a combination thereof.

It is another object of the invention to disclose the method as defined above, additionally comprising a step for selecting an object orientation from a group consisting of prone, supine, fetal position, recovery position, and a combination thereof.

It is another object of the invention to disclose the method as defined above, additionally comprising step of selecting a preferred scanning quality of object from a group consisting of sharpness, noise, contrast, artifacts of MRI software, TI, automatic selection and a combination thereof.

It is another object of the invention to disclose the method as defined above, additionally comprising step of selecting a preferred scanning quality according with said tumor characteristics.

It is another object of the invention to disclose the method as defined above, additionally comprising step for selecting a preferred scanning orientation from a group consisting of horizontal plane, perpendicular plane, transverse plane, coronal plane, sagittal plane and any combination thereof.

It is another object of the invention to disclose the method as defined above, additionally comprising step of selecting a preferred scanning type from a group consisting of two-dimensional scanning, three-dimensional scanning.

It is another object of the invention to disclose the method as defined above, additionally comprising step of providing a GUI with an additional save scan option, wherein said save scan option enables said GUI user to save a scanning.

It is another object of the invention to disclose the method as defined above, additionally comprising step of providing a GUI with an additional export option, wherein said export option enables said GUI user to export any data from a scanning to any external program.

It is another object of the invention to disclose the method as defined above, additionally comprising a step of providing a GUI with an additional loading previous scan option, wherein said additional loading previous scan option enables said GUI user to upload previously saved scanning to GUI.

It is another object of the invention to disclose the method as defined above, additionally comprising a step of providing a GUI with an additional scan new object option according to parameters of loaded scan.

It is another object of the invention to disclose the method as defined above, additionally comprising step of providing a GUI with an additional scan compare option, wherein said scan compare option enables said GUI user to compare scanning.

It is another object of the invention to disclose the method as defined above, additionally comprising step of providing a GUI with an additional option of selecting tumor characteristics of object.

It is another object of the invention to disclose the method as defined above, additionally comprising step of selecting a comparative display from a group consisting of compare scanning performed at different time, compare scanning performed at different locations of an object, compare of different objects, and a combination thereof.

It is another object of the invention to disclose the method as defined above, additionally comprising step of selecting tumor characteristics from a group consisting of small tumor, large tumor, intermediate size tumor, type of tumor, and a combination thereof.

It is another object of the invention to disclose the method as defined above, additionally comprising step of selecting a type of tumor from a group consisting of carcinoma, sarcoma, lymphoma, germ cell tumor, blastoma, melanoma, glyoma, hepatoma and a combination thereof.

It is another object of the invention to disclose an MRI computerized operating system 101 for scanning an object by an MRI. System 101 comprising a display operatively connected to an MRI; object to be scanned within said MRI; and, a GUI presented on said display for interacting with said MRI. Wherein at at least one first region of the GUI, graphic visualization options relating to the object and scanning characteristics of the said object are displayed. Visualization options are selectable from a group consisting of type of object, object orientation, preferred scanning region of object, preferred scanning quality of object, preferred scanning orientation, preferred scanning type, and a combination thereof. Display, is adapted via the GUI to enable selection of the object characteristics and scanning characteristics; the MRI is adapted by means of interacting with the display for scanning the object according to the object characteristics and scanning characteristics.

It is another object of the invention, to disclose the system 101 as defined above, wherein type of object that we are working with is selected according to the standard biological classification of organisms from a group consisting of kingdom, phylum, class, order, family, genus, species, subspecies and a combination thereof.

It is another object of the invention, to disclose the system 101 as defined above, wherein type of object is selected from a group consisting of object identification number, type of experiment, type of disease, and a combination thereof.

It is another object of the invention, to disclose the system 101 as defined above, wherein object orientation is selected from a group consisting of prone, supine, fetal position, recovery position, and a combination thereof.

It is another object of the invention, to disclose the system 101 as defined above, wherein preferred scanning quality of object is selected from a group consisting of sharpness, noise, contrast, artifacts of MRI software, TI and a combination thereof.

It is another object of the invention, to disclose the system 101 as defined above, wherein preferred scanning quality is selected automatically or non-automatically according to said tumor characteristics.

It is another object of the invention, to disclose the system 101 as defined above, wherein preferred scanning orientation is selected from a group consisting of horizontal plane, perpendicular plane, transverse plane, coronal plane, sagittal plane and any combination thereof.

It is another object of the invention, to disclose the system 101 as defined above, wherein preferred scanning type is selected from a group consisting of two-dimensional scanning, three-dimensional scanning and a combination thereof.

It is another object of the invention, to disclose the system 101 as defined above, wherein GUI comprises at least one icon of a save scan option, wherein said save scan option enables the GUI user to save a scanning.

It is another object of the invention, to disclose the system 101 as defined above, wherein GUI comprises at least one icon of an export option, wherein said export option enables the GUI user to export any data from a scanning to any external program.

It is another object of the invention, to disclose the system 101 as defined above, wherein GUI comprises at least one icon of a loading previous scan option, wherein said additional loading previous scan option enables the GUI user to upload previously saved scanning to the GUI.

It is another object of the invention to disclose the system 101 as defined above, wherein GUI comprises at least one icon of scan new object option according to parameters of loaded scan.

It is another object of the invention, to disclose the system 101 as defined above, wherein GUI comprises at least one icon of a scan compare option, wherein said scan compare option enables said GUI user to compare scanning.

It is another object of the invention, to disclose the system 101 as defined above, wherein GUI comprises at least one icon of a tumor characteristic selection option.

It is another object of the invention, to disclose the system 101 as defined above, wherein GUI comprises at least one icon of a presentation mode of a scanning.

It is another object of the invention, to disclose the system 101 as defined above, wherein presentation mode is selected from a group consisting of single image display, multi-image display, thumbnail display, comparative display and a combination thereof, and displaying the same on said display.

It is yet another object of the invention, to disclose the system 101 as defined above, wherein a comparative display is selected from a group consisting of compare scanning performed at different time, compare scanning performed at different locations of an object, compare of different objects, and a combination thereof.

It is still another object of the invention, to disclose the system 101 as defined above, wherein a tumor characteristics are selected from a group consisting of small tumor, large tumor, intermediate size tumor, type of tumor, and a combination thereof.

The type of tumor is selected, according to one embodiment of the invention, from a group consisting of carcinoma, sarcoma, lymphoma, germ cell tumor, melanoma, glyoma, hepatoma blastoma and a combination thereof.

### BRIEF DESCRIPTION OF THE FIGURES

In order to understand the invention and to see how it may be implemented in practice, a few preferred embodiments will now be described, by way of non-limiting example only, with reference to be accompanying drawings, in which:

FIG. 1 presents a flow chart of a method for scanning using an MRI;

FIG. 2 presents a GUI for selection of action to be performed, the action can be either one of starting a new scan, viewing and comparing scans, loading previously scanned scan and exporting scan;

FIG. 3 presents a GUI for selecting object identification, selection can be in accordance of experiment, sample ID, research and type of object to be scanned;

FIG. 4 presents a GUI for selecting object position and tumor location, object position can be either prone or supine and tumor location can be either head body or whole body;

FIG. 5 presents a GUI for selecting image quality, image quality is selected in accordance with tumor size dimensionality of scanning;

FIG. 6 presents a display for preview results, preview is correlated with scanning type;

FIG. 7-10 presents different options for displaying MRI scanning results;

FIG. 11 presents a GUI for exporting MRI scanning; and,

FIG. 12 presents an MRI computerized operating system 101 for scanning an object.

### DETAILED DESCRIPTION OF THE PREFERRED EMBODIMENTS

The following description is provided so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide methods and systems for operating an MRI.

The present invention provides a method for scanning using MRI with a user-friendly software interface which involves a non-MRI terminology. Method comprising steps selected inter alis from (a) providing a display, MRI, and an object 105 to be scanned in MRI, (b) connecting the display to the MRI, (c) displaying the user-friendly interface on the display, (d) displaying on the interface options for characterizing both object 105 and the desirable scanning, (e) selecting from the option the scan to be performed on object 105, (f) scanning object 105 by using the MRI according to the selection of scanning options, (g) optionally, presenting the scanning results on the display.

The present invention also provides a system 101 for operating MRI with a user-friendly software interface-GUI which involves non-MRI terminology. System 101 comprising a display operatively connected to an MRI, an object 105 to be scanned within the MRI and a friendly interface displayed on display for operating the MRI. User interface includes options for selecting characterizations for object 105 and for the desired scanning of object 105, these requirements are than used to operate the MRI.

It is appreciated that the conventional operation of an MRI, requires knowledge directly of the MRI parameters. Thus, the present invention provides a step-by-step “push-button” interface to simplify MRI operation, where no prior MRI knowledge is required and a non-MRI terminology is used, operation focuses on the “biological target” and not the nuances of MRI/imaging. The present invention includes an icon-based interface for setting-up and running an MRI study, the software interface is user friendly and there is an intuitive workflow. The aim of using a non-MRI terminology is achieved by using optimized, pre-programmed protocols.

Embodiments of the present invention automatically references an extensive library of pre-programmed sequences and imaging protocols to automatically generate appropriate imaging sessions. The protocols and imaging can be updated using the GUI.

The terms “magnetic resonance device” (MRD) and “MRI” interchangeably refers hereinafter to any Magnetic Resonance Imaging (MRI) device, any Nuclear Magnetic Resonance (NMR) spectroscope, any Electron Spin Resonance (ESR) spectroscope, any Nuclear Quadruple Resonance (NQR) or any combination thereof. It is herein acknowledged that embodiments of the present invention are adapted for use with any combination of the aforementioned MRD devices.

The term “GUI” refers herein after to a graphical user interface, which allows user to interact with an external system using graphical images

The term “thumbnails” refers herein after to a reduced size version of pictures displayed on a screen.

The term “M2 Compact MRI system” refers herein after to a compact MRI system specially designed for scanning object 105 in size of a rat or smaller and is used in research labs.

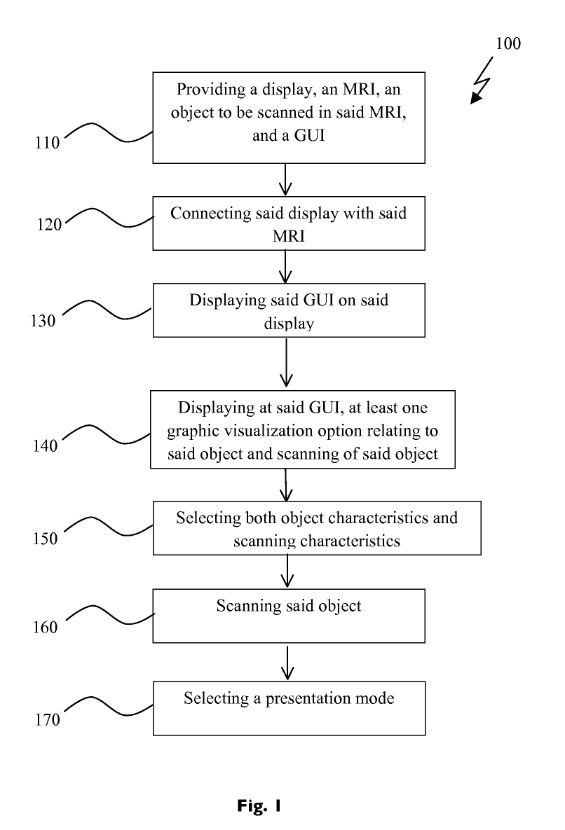

Reference is made to FIG. 1, which shows in a non-limiting manner a method 100 for operating a system of MRI 102 using a user friendly interface (i.e., the GUI). Method 100 includes, inter alis, providing a display, an MRI 102, an object 105 to be scanned by the MRI 102 and a GUI 104 (step 110), connecting a display with MRI 102 (step 120), displaying a GUI 104 on a display (step 130), displaying at GUI 104 a graphical visualization options relating to object 105 and scanning of object 105 (step 140), selecting using GUI 104 the object characteristics and the scanning characteristics (step 150), scanning object 105 according to the selected options (step 160), optionally, selecting a presentation mode (step 170).

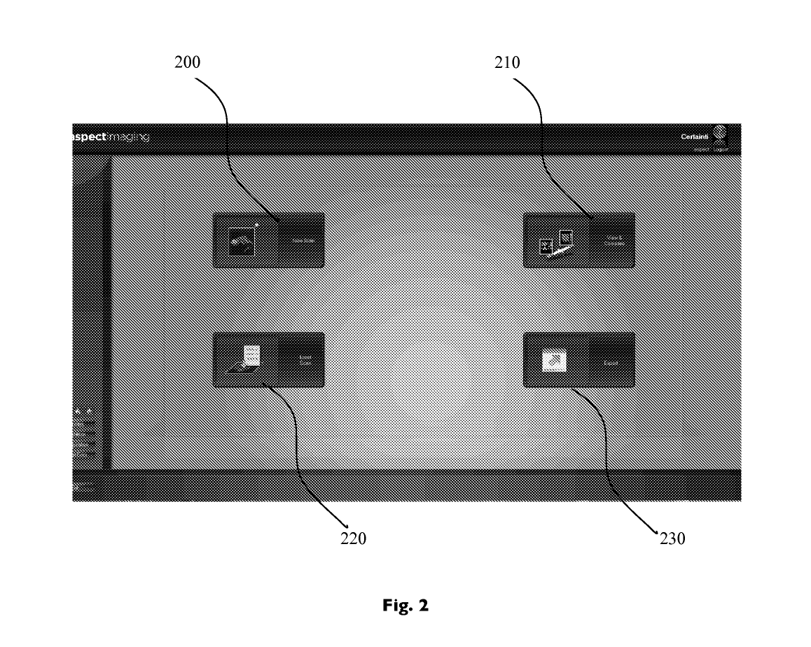

Reference is now made to FIG. 2, which presents in a non-limiting manner an additional step for the method 100 of selecting options for operating GUI 104. Possible selections are for example starting a new scan 200, view and compare older scans 210 for selecting older scans of the same object 105 or scans from different objects to view and compare, load previously saved scan 220 for uploading scans which were previously scanned and using same selections to scan a new sample, and exporting a scan 230 for sending scanning to any type of external software or hardware

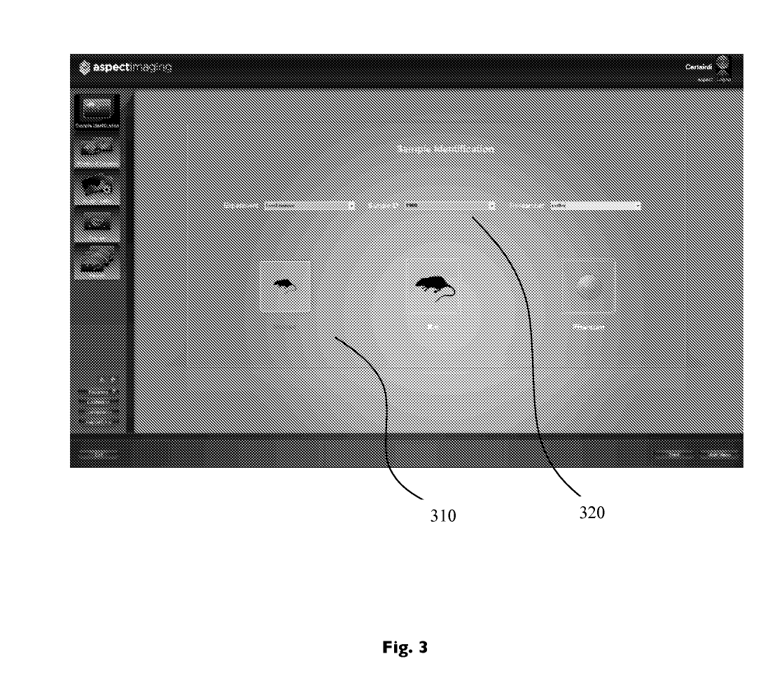

Reference is now made to FIG. 3, which depict in a non-limiting manner an additional step for a method 100 of selecting GUI 104 options for identifying object 105. Possible options includes for example, selecting between a mouse, rat or phantom (310), in other embodiment of the present invention, selection of type of object 105 includes kingdom, phylum, class, order, family, genus, species, sub-species, and any possible combination. Other possible sample identification options 320 are for example choosing experiment type, sample ID, research, in other embodiment of the present invention another identification option is a type of disease.

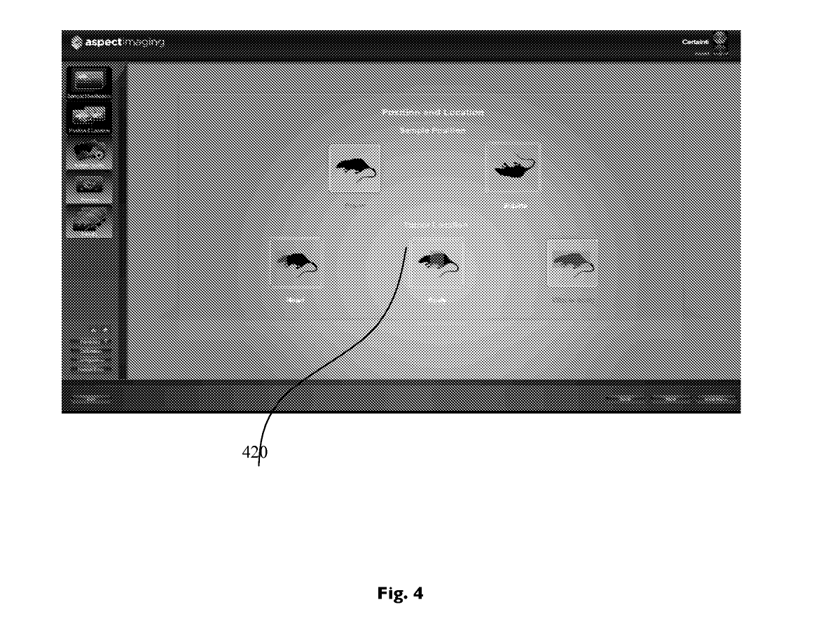

Reference is now made to FIG. 4, which depict in a non-limiting manner an additional step for a method 100 of selecting for the positioning the object 410 and for locating the tumor 420. Object 105 position is chosen for example to be prone or supine. The scan location is chosen to be for example either one of head, body or whole body.

In other embodiment of the present invention selection of object 105 positioning is for example either one of prone, supine, fetal position, recovery position and any combination.

In other embodiment of the present invention, selecting tumor characteristics is done by GUI 104. Tumor characteristics are for example small tumor, large tumor, intermediate tumor, type of tumor and any combination. In other embodiment of the present invention, type of tumor is for example either one of carcinoma, sarcoma, lymphoma, melanoma, glyoma, hepatoma, germ cell tumor blastoma and any combination.

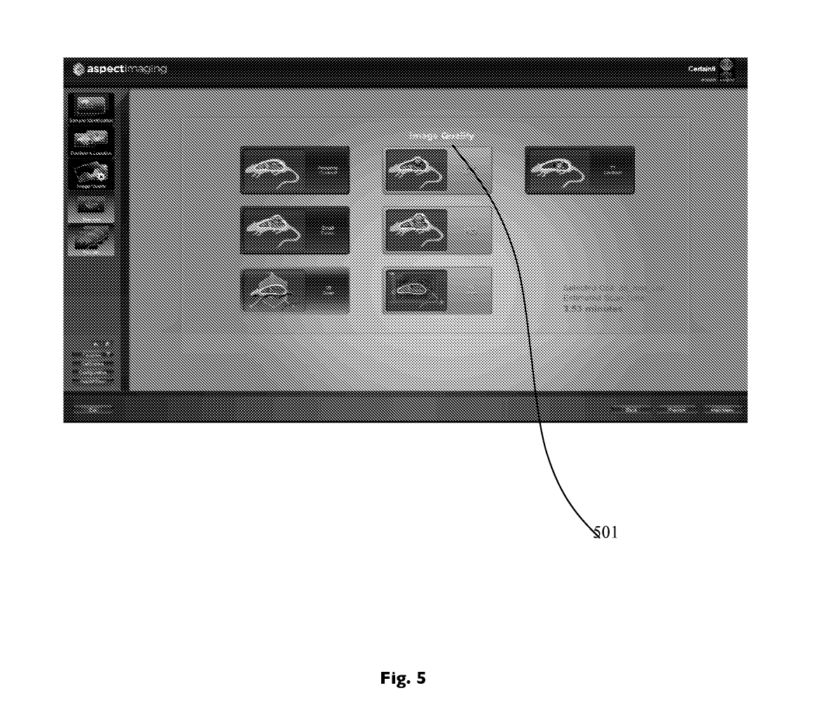

Reference is now made to FIG. 5, which displays in a non-limiting manner an additional step for a method 100 of selecting image quality 501. Image quality is determined for example according to any one of the options automatic selection, small tumor, large tumor, three dimensional scan, two dimensional scan and any combination.

In other embodiment of the present invention, selecting scanning quality is directly chosen in accordance to selected parameters such as sharpness, noise, contrast, artifacts of MRI 102 software and any combination.

In other embodiment of the present invention, step of selecting scanning orientation is presented. Scanning orientation is for example either one of horizontal plane, perpendicular plane, transverse plane, coronal plane, sagittal plane and any combination

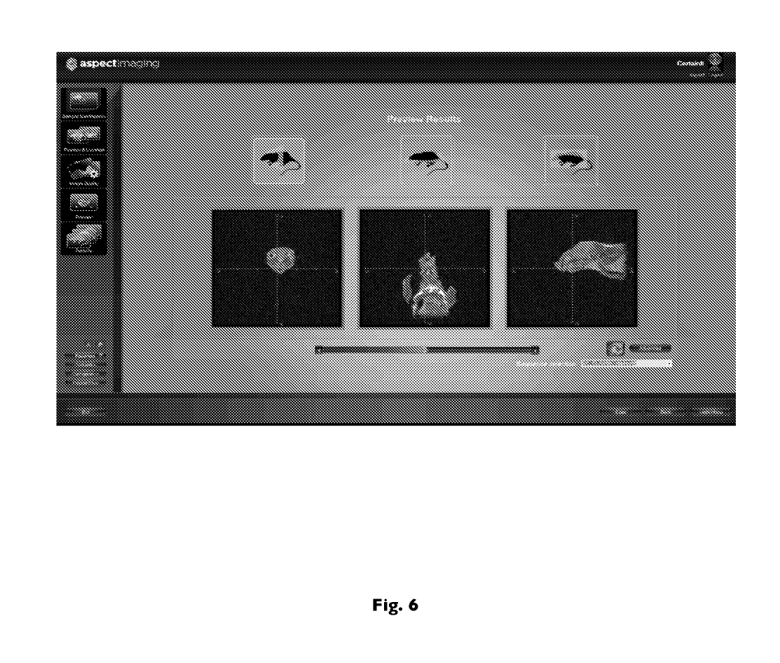

Reference is now made to FIG. 6, which presents in a non-limiting manner an additional step for a method 100 of displaying different preview results according to scanning orientation.

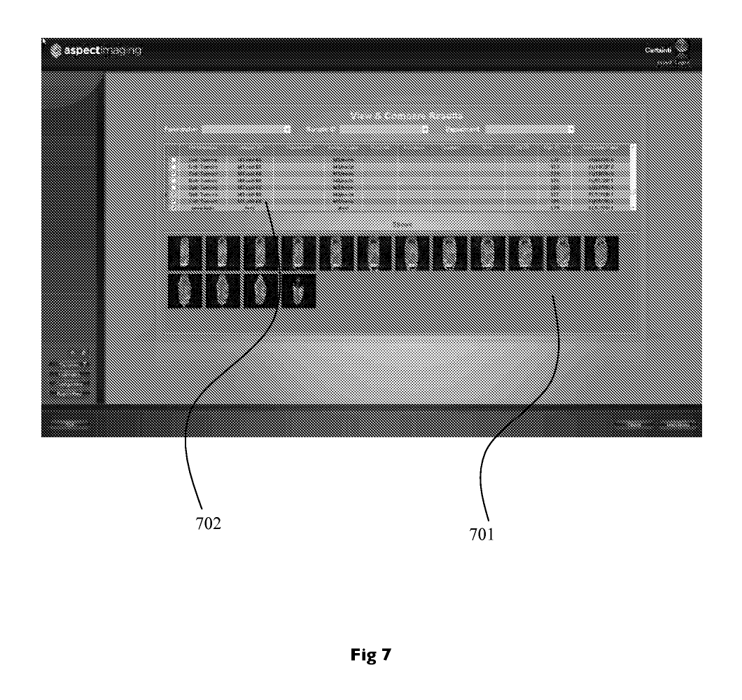

Reference is now made to FIG. 7, which shows in a non-limiting manner an additional step for a method 100 of visually comparing 701 between different scans, the scans to be compared are presented in another area 702 of GUI 104.

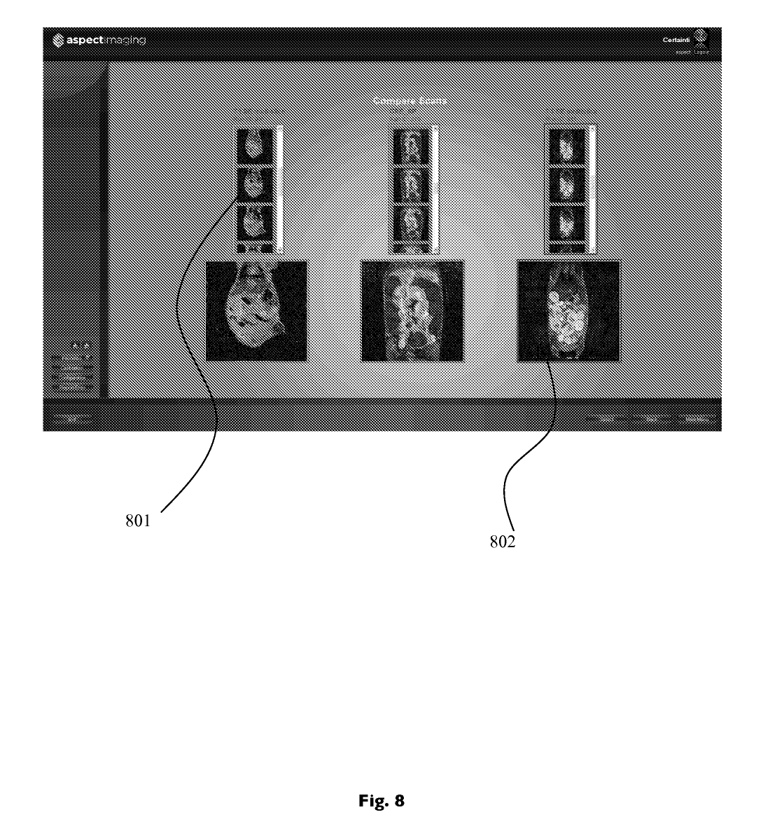

Reference is now made to FIG. 8, which presents in a non-limiting manner a method for visually comparing different sets of scans. In this method each set of scans 801 is displayed separately, and an option is presented for choosing a specific scan 802 from each set and comparing scans from different sets.

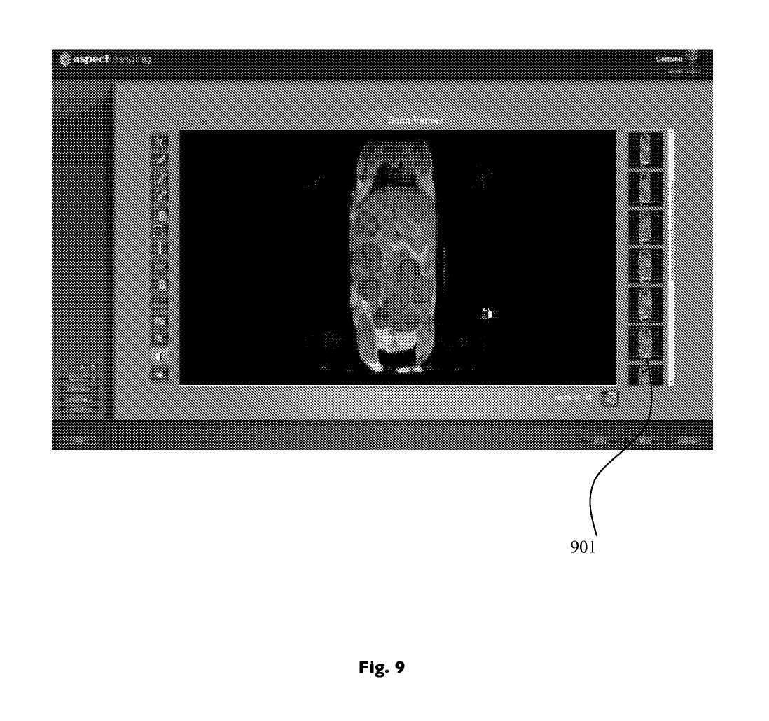

Reference is now made to FIG. 9, which presents in a non-limiting manner a method for presenting full screen view of a single scan is presented. It is possible in this method to show a set of scans in thumbnail, from which, GUI 104 user chooses a scan to present in full screen mode.

In other embodiment of the present invention, a method for displaying scans is presented in a non-limiting manner. Displaying options are selected for example from the options single image display, multi-image display, thumbnail display and any combination.

In other embodiment of the present invention, a method for comparing different scans is presented. Comparing can be for example either one of compare scanning performed at different location of object 105, compare between scans performed at different times or compare between scans of different objects. Compare can be also any combination of the above.

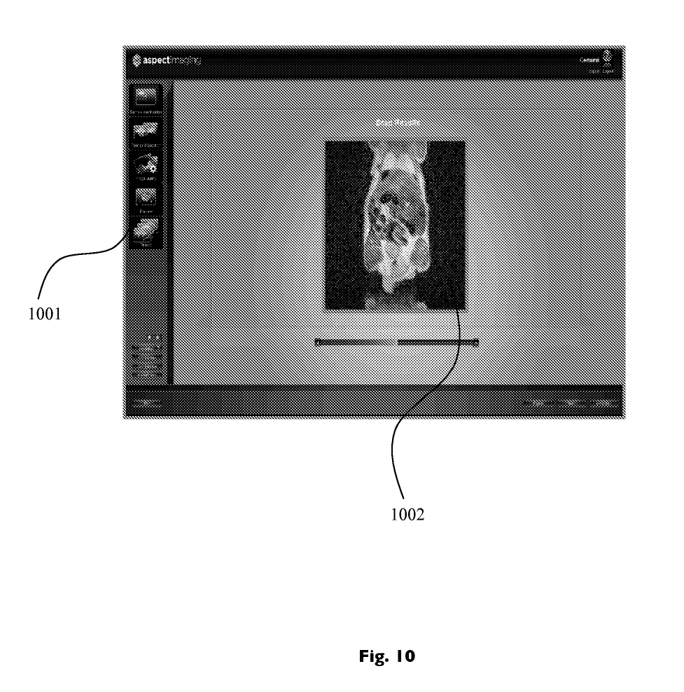

Reference is now made to FIG. 10, which demonstrate in a non-limiting manner an additional step for a method 100 of presenting full screen view of a single scan. Single scan is loaded at a menu 1001, and is presented at a single scan view 1002.

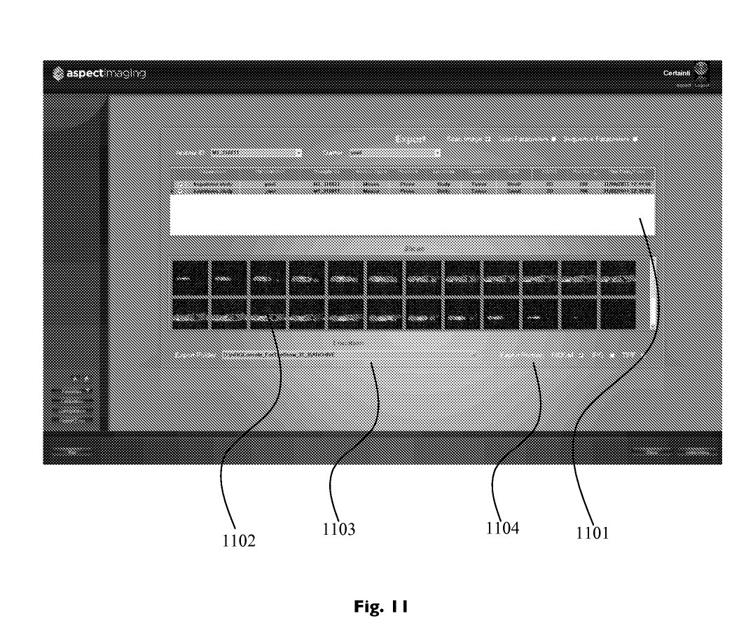

Reference is now made to FIG. 11, which presents in a non-limiting manner an additional step for a method 100 of exporting scans to any external program. Exporting is performed by selecting scans to be exported at one area 1101 of the screen, following the selection, slices of scans are than visually presented as thumbnails 1102. Furthermore, the method comprises step of selecting export folder 1103 to which scans will be exported, and export format 1104, for selecting the image type of the selected scans.

### EXAMPLE 1

This example is provided in a non-limiting manner to illustrate one scope of the invention, wherein method 100 includes, inter alis, steps as follows (i) providing a 24 inch size screen equipped with the commercially available operating system named windows XP, with screen resolution of 1920×1200, the commercially available compact MRI system named M2 Compact MRI system, a rat and a GUI 104; (ii) connecting the screen to the M2 and displaying GUI 104 on the display; (iii) presenting on GUI 104 options for selecting type of experiment, a mouse or rat option and the identification number of the rat; (iv) presenting on GUI 104 additional option for selecting whether the rat is in prone position or in supine position; (v) presenting on GUI 104 additional option for selecting tumor location of the rat to be for example either one of head, body or whole body; (vi) presenting on GUI 104 an additional option for selecting image quality for example according to large tumor or small tumor; (vii) presenting on GUI 104 an additional option for selecting two-dimensional scan or three-dimensional scan; (viii) scanning the rat with accordance to the chosen options; (ix) presenting on a display an option for preview result; and, (x) presenting on GUI 104 additional option for comparing scans.

It is possible in the current example to additionally compare scanning performed either at different times on the same rat for the purpose of identifying tumor change. It is acknowledged that the example depicted is mainly used for study focused on animal anatomy and tumor localization, tumor angiography using contrast agents and longitudinal study.

### EXAMPLE 2

Another example is presented in a non-limiting manner to illustrate the scope of the invention, wherein method 100 includes an MRI adapted to scan humans. Scans are used not only for research purposes but also for diagnostic purposes. In the current example, the method 100 additionally comprising a step of selecting human gender, age and medical history. Additionally, the method comprises a set of steps useful for acquiring treatment history and suggesting future treatment in accordance with scanning results.

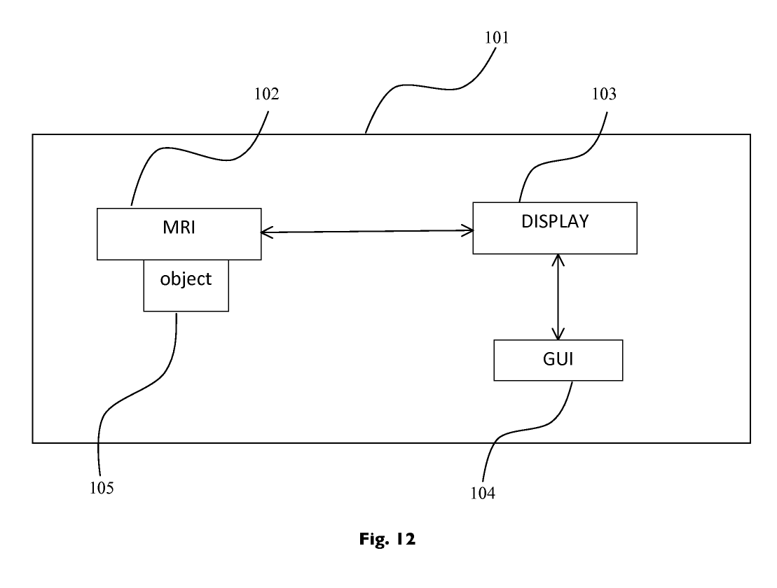

The MRI computerized operating system is now discussed. Reference is made again for FIG. 12, which presents in a non-limiting manner an MRI 102 computerized operating system for scanning object 105. System comprises (i) an MRI 102; (ii) a display 103; (iii) a GUI 104; and, (iv) object 105 to be scanned by MRI 102. Display 103 is operatively connected to MRI, and GUI 104 is presented on display 103. Additionally, GUI 104 presents graphic visualization options relating to object 105 and scanning characteristics of object 105 are displayed; furthermore, display 103 is adapted via GUI 104 to enable selection of said object characteristics and scanning characteristics; and, MRI 102 is adapted by means of interacting with display 103 for scanning object 105 according to object 105 characteristics and scanning characteristics.

Reference is now made again to FIG. 2, which presents in a non-limiting manner selection options for operating GUI 104, it is possible for selections to be for example starting a new scan 200 for activation of MRI 102 to perform a new scan, view and compare older scans 210 for selecting older scans of the same object 105 or scans from different options to view and compare, load previously saved scan 220 for uploading scans which were previously scanned, and exporting a scan 230 for sending scanning to any type of external software or hardware

Reference is now made again to FIG. 3, which illustrates in a non-limiting manner a GUI 104 selection options for sample identification, possible options include, selecting between a mouse, rat or phantom (310). In other embodiment of the present invention, selection of type of object 105 includes for example kingdom, phylum, class, order, family, genus, species, sub-species, and any possible combination. Other possible sample identification options 320 are choosing experiment type, sample ID, research, in other embodiment of the present invention another identification option may be a type of disease.

Reference is now made again to FIG. 4, which presents in a non-limiting manner a GUI 104 selection option for the object position 410 and for the tumor location 420. Object position may be for example prone or supine, and tumor location maybe head body or whole body.

In other embodiment of the present invention, GUI 104 selection options of the object position may be for example either one of prone, supine, fetal position, recovery position and any combination.

In other embodiment of the present invention, a system 101 additionally comprises in a non-limiting manner an option for selecting tumor characteristics. Tumor characteristics can be for example either one of small tumor, large tumor, intermediate tumor, type of tumor and any combination. Furthermore, type of tumor can be either one of carcinoma, sarcoma, lymphoma, melanoma, glyoma, hepatoma, germ cell tumor blastome and any combination.

Reference is now made again to FIG. 5, which presents in a non-limiting manner a GUI 104 selection option for the image quality 501, where image quality options can be for example chosen to be either one of automatic selection, small tumor, large tumor, three dimensional scan, two dimensional scan and any combination.

In other embodiment of the present invention, a system 101 additionally comprises in a non-limiting manner, scanning quality option, selected for example to be either one of sharpness, noise, contrast, artifacts of MRI 102 software and any combination.

In other embodiment of the present invention, a system 101 additionally comprises in a non-limiting manner an option for selecting scanning orientation, where scanning orientation can be for example either one of horizontal plane, perpendicular plane, transverse plane, coronal plane, sagittal plane and any combination

Reference is now made again to FIG. 6 which presents in a non-limiting manner an option to display different preview results according to scanning orientation.

Reference is now made again to FIG. 7, which presents in a non-limiting manner an option for visually comparing 701 between different scans, where scans to be compared are presented in another area 702 of a GUI 104.

Reference is now made again to FIG. 8, which presents in a non-limiting manner a compare option between different scans. Each set of scans 801 is presented separately, and an option is presented for choosing a specific scan 802 from each set and comparing scans from different sets.

Reference is now made again to FIG. 9, which presents in a non-limiting manner a view option for viewing a scan on a full screen. Additionally, a set of scan is presented in thumbnail, from which, a GUI 104 user chooses a scan to present in full screen mode.

In other embodiment of the present invention, a system 101 additionally comprises in a non-limiting manner different options for displaying scans. Options can be selected for example to be either one of single image display, multi-image display, thumbnail display and any combination.

In other embodiment of the present invention, a system 101 additionally comprises in a non-limiting manner an option for compare of different scans. Compare can be either one of comparing scans performed at different location of object 105, compare between scans performed at different times, and compare between scans of different objects.

Reference is now made again to FIG. 10 which presents in a non-limiting manner an option for presenting full screen view of a single scan is presented. Single scan is loaded at menu 1001, and is presented at a single scan view 1002.

Reference is now made again to FIG. 11, which presents in a non-limiting manner an export option for exporting scans to any external program. Exporting is performed by selecting scans to be exported at one area 1101 of screen; slices of scans are than visually presented as thumbnails 1102. Furthermore, the method comprises step of selecting export folder 1103 and export format 1104.

### EXAMPLE 3

This example is presented in a non-limiting manner to illustrate the scope of the invention, wherein in one embodiment of the current invention the system comprises (i) a 24 inch size screen equipped with the commercially available operating system named windows XP, with screen resolution of 1920×1200; (ii) the commercially available compact MRI system named M2 Compact MRI system; (iii) a rat; and (iv) a GUI 104. It is possible to connect the screen to the M2 and to display a GUI on the display. Additionally, GUI 104 presents options for selecting type of experiment, the object identity to be a mouse or rat, and the identification number of the mouse or rat. In this system GUI 104 additional comprises an option for selecting whether the rat is in prone position or in supline position. It is also possible to select via GUI 104 tumor location of the rat to be either one of head, body or whole body. Additionally, GUI 104 comprises an option for selecting image quality in accordance with the options of large tumor, small tumor. It is possible to select via GUI 104 scanning of type two-dimensional or three-dimensional. In this system, a rat is scanned with accordance to the chosen options. Following scanning of a rat, GUI 104 comprises an option for preview results and comparing results in accordance with scanning orientation.

In the present embodiment of the current invention, compare scans is of scans performed at different times of the same rat, for purpose of identifying tumor change. The purpose of the scanning is study focuses on animal anatomy and tumor localization, tumor angiography using contrast agents and longitudinal study.

### EXAMPLE 4

This example is presented in a non-limiting manner to illustrate the scope of the invention, wherein in one embodiment of the current invention a GUI is compatible to operate an MRI adapted to scan humans, scans are used not only for research purposes but also for diagnostic purposes. In the current example, the system 101 comprises icons for specifying human gender, age and medical history. It is also adapted to acquire treatment history and produce suggestions for future treatments in accordance with scanning results.

Examples of various features/aspects/components/operations have been provided to facilitate understanding of the disclosed embodiments of the present invention. In addition, various preferences have been discussed to facilitate understanding of the disclosed embodiments of the present invention. It is to be understood that all examples and preferences disclosed herein are intended to be non-limiting.

Although selected embodiments of the present invention have been shown and described individually, it is to be understood that at least aspects of the described embodiments may be combined.

Although selected embodiments of the present invention have been shown and described, it is to be understood the present invention is not limited to the described embodiments. Instead, it is to be appreciated that changes may be made to these embodiments without departing from the principles and spirit of the invention, the scope of which is defined by the claims and the equivalents thereof.

What is claimed is:

1. A method for scanning using an MRI, comprising:
a. providing a display, MRI, object to be scanned in said MRI, and a GUI;
b. connecting said display with said MRI;
c. displaying said GUI on said display;
d. displaying at at least one first region of said GUI, graphic visualization options relating to said object and scanning of object, said visualizations are selected from a group consisting of type of object, object orientation, preferred scanning region of object, preferred scanning quality of object, preferred scanning orientation, preferred scanning type, and a combination thereof;
e. selecting said object characteristics and scanning characteristics via said GUI;
scanning said object by means of said MRI in accordance with said determined characteristics; and
f. optionally, selecting a presentation mode of said scanning from a group consisting of single image display, multi-image display, thumbnail display, comparative display and a combination thereof, and displaying the same on said display.

2. The method according to claim 1, wherein said method additionally comprising in step (d), at least one step selected from a group consisting of:
a. selecting a type of object from a group consisting of kingdom, phylum, class, order, family, genus, species, subspecies and a combination thereof;
b. selecting a type of object from a group consisting of object identification number, type of experiment, type of disease, and a combination thereof;
c. selecting a object orientation from a group consisting of prone, supine, fetal position, recovery position, and a combination thereof;
d. selecting a preferred scanning quality of object from a group consisting of sharpness, noise, contrast, artifacts of MRI software, TI, automatic selection and a combination thereof;
e. selecting a preferred scanning quality according with said tumor characteristics;
f. selecting a preferred scanning orientation from a group consisting of horizontal plane, perpendicular plane, transverse plane, coronal plane, sagittal plane and any combination thereof; and,
g. selecting a preferred scanning type from a group consisting of two-dimensional scanning, three-dimensional scanning and a combination thereof.

3. The method according to claim 1, wherein said method additionally comprising at least one step selected from a group consisting of:
a. providing a GUI with an additional save scan option, wherein said save scan option enables said GUI user to save a scanning;
b. providing a GUI with an additional export option, wherein said export option enables said GUI user to export any data from a scanning to any external program;
c. providing a GUI with an additional loading previous scan option, wherein said additional loading previous scan option enables said GUI user to upload previously saved scanning to said GUI;
d. providing a GUI with an additional scan compare option, wherein said scan compare option enables said GUI user to compare scanning; and
e. providing a GUI with an additional option of selecting tumor characteristics of object.

4. The method according to claim 3, wherein said method additionally comprising in step (c) an additional step of scanning a new object with parameters of said loaded scan.

5. The method according to claim 1, additionally comprising step of selecting a comparative display from a group consisting of compare scanning performed at different time, compare scanning performed at different locations of an object, compare of different objects, and a combination thereof.

6. The method according to claim 2, additionally comprising step of selecting a tumor characteristics from a group consisting of small tumor, large tumor, intermediate size tumor, type of tumor, and a combination thereof.

7. The method according to claim 3, additionally comprising step of selecting a tumor characteristics from a group consisting of small tumor, large tumor, intermediate size tumor, type of tumor, and a combination thereof.

8. The method according to claim 6, additionally comprising step of selecting a type of tumor from a group consisting of carcinoma, sarcoma, lymphoma, melanoma, glyoma, hepatoma, germ cell tumor, blastoma and a combination thereof.

9. An MRI computerized operating system for scanning an object by an MRI, said system comprising:
a. a display operatively connected to an MRI;
b. object to be scanned within said MRI; and
c. a GUI presented on said display for interacting with said MRI,
wherein at at least one first region of said GUI, graphic visualization options relating to said object and scanning characteristics of said object are displayed, said visualization options selectable from a group consisting of type of object, object orientation, preferred scanning region of object, preferred scanning quality of object, preferred scanning orientation, preferred scanning type, and a combination thereof; said display, is adapted via said GUI to enable selection of said object characteristics and scanning characteristics;
said MRI is adapted by means of interacting with said display for scanning said object according to said object characteristics and scanning characteristics.

10. The system according to claim 9, wherein one of the following is held true:
a. type of object is selected from a group consisting of kingdom, phylum, class, order, family, genus, species, subspecies and a combination thereof;
b. type of object is selected from a group consisting of object identification number, type of experiment, type of disease, and a combination thereof;
c. object orientation is selected from a group consisting of prone, supine, fetal position, recovery position, and a combination thereof;
d. preferred scanning quality of object is selected from a group consisting of sharpness, noise, contrast, artifacts of MRI software, TI and a combination thereof;
e. preferred scanning quality is selected automatically or non-automatically according to said tumor characteristics;
f. preferred scanning orientation is selected from a group consisting of horizontal plane, perpendicular plane, transverse plane, coronal plane, sagittal plane and any combination thereof; and
g. preferred scanning type is selected from a group consisting of two-dimensional scanning, three-dimensional scanning and a combination thereof.

11. The system according to claim 9, wherein said GUI comprises at least one icon selected from a group consisting of:
a. a save scan option, wherein said save scan option enables said GUI user to save a scanning;
b. an export option, wherein said export option enables said GUI user to export any data from a scanning to any external program;
c. a loading previous scan option, wherein said additional loading previous scan option enables said GUI user to upload previously saved scanning to said GUI;
d. a scan compare option, wherein said scan compare option enables said GUI user to compare scanning;
e. a tumor characteristic selection option object; and
f. a presentation mode of a scanning.

12. The system according to claim 11, wherein said GUI additionally comprising at least one icon for scan a new object using parameters of said loaded scan.

13. The system according to claim 11, wherein presentation mode is selected from a group consisting of single image display, multi-image display, thumbnail display, comparative display and a combination thereof, and displaying the same on said display.

14. The system according to claim 13, wherein said a comparative display is selected from a group consisting of compare scanning performed at different time, compare scanning performed at different locations of an object, compare of different objects, and a combination thereof.

15. The system according to claim 10, wherein a tumor characteristics are selected from a group consisting of small tumor, large tumor, intermediate size tumor, type of tumor, and a combination thereof.

16. The system according to claim 11, wherein a tumor characteristics are selected from a group consisting of small tumor, large tumor, intermediate size tumor, type of tumor, and a combination thereof.

17. The system according to claim 15, wherein a type of tumor is selected from a group consisting of carcinoma, sarcoma, lymphoma, melanoma, glyoma, hepatoma, germ cell tumor blastoma and a combination thereof.
